(19) United States
(12) Patent Application Publication
(10) Pub. No.: US 2003/0162179 A1
(43) Pub. Date: Aug. 28, 2003

(54) Fabrication, performance testing, and screening of three dimensional arrays of materials

(75) Inventors: Radislav Alexandrovich Potyrailo, Niskayuna, NY (US); James Norman Cawse, Pittsfield, MA (US)

Correspondence Address: General Electric Company, CRD Patent Docket Rm 4A59, Bldg. K-1 -Salamone, P.O. Box 8, Schenectady, NY, 12301, US

(73) Assignee: General Electric Company

(21) Appl. No.: 10/083,727

(22) Filed: Feb. 27, 2002

(51) Int. Cl. C12Q 1/68; B05D 3/00; G01N 33/53; G06F 19/00; G01N 33/48; G01N 33/50; C12M 1/34

(52) U.S. Cl. 435/6; 435/287.2; 427/2.11; 702/20

(57) ABSTRACT

The present invention relates to methods for the fabrication, performance testing and screening of combinatorial libraries of materials arranged as three dimensional arrays. The invention describes the generation and screening of three dimensional arrays by depositing a plurality of samples onto at least one substrate at discrete and defined positions in a three dimensional format such that each sample is isolated by the substrate from the other samples, and wherein each sample is defined by its (x, y, and z) coordinate, collecting analytical data from the sample array, correlating the analytical data collected from the array to the position of samples within the array, and analyzing the analytical data for a parameter of interest. The array may be performance tested for stability to chemical degradation, environmental stress, or other factors. Also described is the use of techniques such as confocal and multi-photon microscopy for the measurement and analysis of samples in the array.

### BACKGROUND OF THE INVENTION

The present invention relates to methods for the fabrication, performance testing, and screening of three dimensional (3D) arrays of materials, and more particularly to three dimensional arrays of combinatorial libraries.

Combinatorial chemistry involves the analysis of a plurality of samples, often termed a library, wherein each of the samples in the library is potentially a unique compound. Combinatorial chemistry allows for simultaneous variance of at least one, and often more than one, reaction parameter such that a systematic variance in sample composition may be explored, thereby yielding a large amount of information about the reaction of interest. For example, combinatorial libraries may include samples generated using identical starting reagents, but at a different processing temperature. Alternatively, libraries may include samples in which one reagent is systematically varied so as to generate an array of similar, but chemically distinguishable, products. Libraries may also be generated from samples in which several reaction parameters (i.e. temperature and reagent profile) are varied according to a predetermined reaction set-up.

A primary advantage of combinatorial chemistry is the ability to generate and analyze large numbers of reactions quickly, so as to determine how different variables affect the reaction being explored. To analyze large numbers of reaction formats, combinatorial libraries preferably utilize multiple small-scale reactions to minimize cost and reagent waste. Still, the time to process and analyze the number of samples required for a detailed combinatorial analysis can be a limiting factor in the technology.

Generally, samples in a combinatorial library are arranged in an array format. Both one and two dimensional arrays materials have been employed for combinatorial screening assays. For example, methods for the fabrication and testing of one-dimensional arrays of coatings for barrier properties (i.e. resistance to chemical and/or physical wearing) has been described (R. Potyrailo, U.S. patent application Ser. No. 09/681,432, filed Apr. 4, 2001). In another approach, one dimensional arrays comprising ten regions each with four different fluorophores were generated along the length of an optical fiber and analyzed using optical time-of-flight measurements (Prince, B. M. et al., Analytical Chemistry, 2001, 73: 1007-1015). One dimensional arrays have also been applied to analytical analysis of peptide binding, wherein a library of peptides was synthesized on a cotton thread and evaluated for binding to streptavidin by fluorescence spectroscopy (Schwabacher, A. W., et al., J. Am. Chem. Soc., 1999, 121: 8669-8670).

For two dimensional arrays, materials and/or processing parameters are introduced in two dimensions. The variations in the parameters of the array may either by gradient (Hanak, J. J., J. Materials Science, 1970, 5: 964-971) or discrete (Xiang, X.-D., et al., Science, 1995, 268: 1738-1740). Once the array is generated, each individual region may be tested for the properties of interest. Thus, a well-known example of a two dimensional array is a 96-well microtiter plate. Other formats for two dimensional arrays have also been described. For example, a 69 element array comprising catalyst-containing sol-gel solutions was used for screening amorphous microporous mixed oxides as hydrogenation/oxidation catalysts by emissivity-corrected IR-thermographic imaging (Hozwarth, A., et al., Angew. Chem. Int. Ed., 1998, 37: 2644-2647). Also, triangular arrays have been described. For example, using a triangular format with 15 elements (or reaction regions) per side, an array of three different metals on a quartz wafer with a different catalyst deposited at each of 120 array positions was generated. Each array position had a diameter of 1.5 mm and a thickness of 100 nm (Cong, P., et al., Angew. Chem. Int. Ed., 1999, 38: 484-488).

Still, in the fabrication of two dimensional arrays, the number of elements in the array often cannot be increased because of the limitations of the element size and fixed substrate layout. For example, during performance testing, a testing set-up such as an environmental chamber, heater, or radiation source can accommodate only a certain size or geometry of array for testing. Furthermore, by being able to vary reaction parameters in a third dimension, the amount of information in an array can be greatly increased. For example, using a format with ten variant components (or variations of a component) per dimension, a one dimensional array includes 10 potential reaction regions, a two dimensional array includes 100 potential reaction regions, and a three dimensional array includes 1000 potential reaction regions.

Thus, there is a need to develop methods for the fabrication of three dimensional arrays of materials. The arrays may involve a variety of geometries, depending on the aspect of the material to be tested. There is also an unmet need to develop methods for the testing and analysis of three dimensional arrays. Such arrays may include samples dispersed within the interior of a three dimensional structure, or samples distributed on the outer surface of a three dimensional shape. Such arrays provide an increase in throughput for testing and screening of materials for their properties of interest. Additionally, such arrays provide the ability to test parameters using a wide variety of geometric formats.

### SUMMARY OF THE INVENTION

The present invention relates to methods for the fabrication, performance testing and screening of combinatorial libraries arranged as three dimensional arrays. Such three dimensional arrays increase the efficiency and number of available formats which can be used for combinatorial screening. Arrays of the present invention may be performance tested by exposing the array to test for stability to chemical degradation, environmental stress, or other factors. Also described is the use of scanning techniques such as confocal fluorescent microscopy for the measurement and analysis of samples in such arrays.

Thus, in one aspect, the invention comprises a method for the generation and screening of three dimensional arrays comprising: depositing a plurality of samples onto at least one substrate at discrete and defined positions in a three dimensional format such that each sample is isolated by the substrate from the other samples, and wherein each sample is defined by its (x, y, and z) coordinate to generate a three dimensional array of samples; collecting analytical data from the sample array, wherein the analytical data is at least partially defined by its (x, y, and z) coordinate within the sample array; correlating the analytical data collected from the array to the position of samples within the array; and analyzing the analytical data for a parameter of interest.

In another aspect, the invention comprises a method for the generation and performance testing of three dimensional arrays comprising: depositing a plurality of samples onto at least one substrate at discrete and defined positions in a three dimensional format such that each sample is isolated by the substrate from the other samples, and wherein each sample is defined by its (x, y, and z) coordinate to generate a three dimensional array of samples; performance testing the array; collecting data from the performance testing, wherein the data is at least partially defined by its (x, y, and z) coordinate within the sample array; correlating the analytical data collected from the array to the position of samples within the array; and analyzing the analytical data for a parameter of interest.

In yet another aspect, the invention comprises a method for the analysis of three dimensional arrays comprising: applying a scanning probe over a plurality of samples deposited onto at least one substrate at discrete and defined positions in a three dimensional format such that each sample is isolated by the substrate from the other samples, and wherein each sample is defined by its (x, y, and z) coordinate; collecting analytical data from the array, wherein the analytical data is at least partially defined by its (x, y, and z) coordinate within the sample array; correlating the analytical data collected from the array to the position of samples within the array; and analyzing the analytical data for a parameter of interest.

### BRIEF DESCRIPTION OF THE FIGURES

Various features, aspects, and advantages of the present invention will become more apparent with reference to the following description, appended claims, and accompanying drawings, wherein:

FIG. 1 is a schematic representation of a method for the fabrication, performance testing and screening of three dimensional arrays in accordance with an embodiment of the present invention.

FIG. 2 illustrates methods for the fabrication of three dimensional (3D) libraries in accordance with an embodiment of the present invention where (A) shows stacking of a set of two dimensional libraries, and (B) shows a cross-section of a three dimensional library formed by stacking of a set of two dimensional libraries;

FIG. 3 illustrates methods for fabrication of three dimensional libraries by library deposition onto various types of outer surfaces in accordance with an embodiment of the present invention where (A) shows library deposition onto a three dimensional substrate with flat surfaces, and (B) shows library deposition onto a three dimensional substrate with curved surfaces;

FIG. 4 illustrates a three dimensional array generated by introducing multiple samples into different spatial regions of a substrate material in accordance with an embodiment of the present invention wherein (A) shows an array of differently shaped elements distributed in a preformed substrate such as a wax or polymer, (B) shows an array of identically shaped samples distributed in a photopolymerizable substrate and subsequent polymerization of the substrate containing the array by UV light; (C) shows a three dimensional array generated by diffusing materials inward from the faces of a tetrahedron, where diffusion from the bottom face is shown; and (D) shows a three dimensional array generated by diffusing materials inward from the vertices of a tetrahedron, where diffusion from the top vertex is shown;

FIG. 5 illustrates a general schematic of a three dimensional library generated on the surface of a cylindrical substrate in accordance with an embodiment of the present invention;

FIG. 6 shows a fluorescence confocal image of a three dimensional array in accordance with an embodiment of the present invention wherein samples on the most distal surface of a cylindrical array are probed;

FIG. 7 shows a fluorescence confocal image of a three dimensional array in accordance with an embodiment of the present invention wherein samples on the sides of a cylindrical array are probed;

FIG. 8 shows a combined fluorescence confocal image of a three dimensional array in accordance with an embodiment of the present invention wherein samples on the distal, proximal, and side surfaces of a cylindrical array are probed; and

FIG. 9 shows the fluorescence intensity map for the cross-section of one element from a three dimensional array in accordance with an embodiment of the present invention wherein a sample on the proximal surface of a cylindrical array is probed along the line shown in FIG. 8.

### DETAILED DESCRIPTION OF THE INVENTION

The present invention relates to methods for the fabrication, performance testing, and screening of combinatorial libraries arranged as three dimensional arrays. Such three dimensional arrays are useful for performance testing of large numbers of samples, as for example, by testing samples in the array for stability to physical or chemical stress. The use of three dimensional arrays greatly increases the number of samples which can be screened, as well as the available formats which can be used. Samples may be prepared in final form prior to deposition within the array, or may be exposed to reactants upon being applied to the array. The arrays of the present invention may be prepared as solid preformed units which are relatively stable to storage and/or transport. Thus, the three dimensional arrays of the present invention may comprise a self-contained matrix suitable for transport to various environments for performance testing under a wide variety of conditions. Such arrays may comprise diagnostic kits, wherein the samples in the array are selected to provide analytical data with respect to specific chemical or physical stimuli which may be expected to be encountered.

In one aspect, the invention comprises a method for the generation and screening of three dimensional arrays comprising: depositing a plurality of samples onto at least one substrate at discrete and defined positions in a three dimensional format such that each sample is isolated by the substrate from the other samples, and wherein each sample is defined by its (x, y, and z) coordinate to generate a three dimensional array of samples; collecting analytical data from the sample array, wherein the analytical data is at least partially defined by its (x, y, and z) coordinate within the sample array; correlating the analytical data collected from the array to the position of samples within the array; and analyzing the analytical data for a parameter of interest.

In an embodiment, the array comprises multiple two dimensional arrays. In another embodiment, the array comprises samples deposited on the surface of a three dimensional structure. Preferably, samples are deposited on the surface of the three dimensional structure using masking and gradient deposition techniques.

In an embodiment, samples are deposited on the surface of the three dimensional structure by evaporative methods. The evaporative methods may be carried out using lasers, filaments, electron beams, or ion beams. Alternatively, the evaporative methods may be carried out using molecular beam epitaxy.

In an embodiment, samples are deposited on the surface of the three dimensional structure by glow-discharge processes. In a preferred embodiment, the glow-discharge processes may comprise sputtering.

In another embodiment, samples are deposited on the surface of the three dimensional structure by chemical vapor deposition. Preferably, the chemical vapor deposition process comprises photo-enhanced chemical vapor deposition or plasma-enhanced chemical vapor deposition. In an alternate embodiment, samples are deposited on the surface of the three dimensional structure by pulse laser assisted deposition.

In yet another embodiment, samples are deposited on the surface of the three dimensional structure by mechanical deposition. Preferably, the mechanical deposition comprises spraying, spinning, dipping, draining, flow coating, roller coating, pressure-curtain coating, or brushing.

For application of samples to the surface of a three dimensional structure, the samples preferably have a thickness which ranges from 0.1 nm to 1 cm. More preferably, the sample thickness ranges from 1 nm to 1 mm. Even more preferably, the sample thickness ranges from 10 nm to 200 μm.

In an embodiment, the array comprises samples deposited within the interior of a three dimensional substrate. Preferably, exogenous samples may be introduced into a pre-formed substrate. In an embodiment, the samples are diffused into the preformed substrate. In an embodiment, the substrate comprises a porous material such porous alumina.

In an embodiment, materials are diffused inwards from predetermined surface locations on the substrate. Materials may be diffused inwards from the surface planes of said substrate, or in an alternate embodiment, diffused inwards from the vertices of said substrate. In an embodiment, the substrate is tetrahedral. In another embodiment, the substrate is a cylinder. In another embodiment, the substrate is a sphere. In yet another embodiment, the substrate is a polyhedron.

Alternatively, at least some of the samples may be introduced into a partially formed substrate and at least part of the substrate transformed to its final structure after deposition of the samples. Preferably, the partially formed substrate comprises a gel which is polymerized upon application of radiation. Even more preferably, the gel may comprise acrylates, unsaturated polyester and the like.

In an embodiment, the deposited samples are treated to initiate a chemical reaction within the three dimensional array. In an embodiment, the treatment is applied differently to at least some locations of the array. Alternatively, the treatment may be applied in a constant manner to each location of the array.

In an embodiment, the treatment comprises the addition of a chemical agent. Alternatively, and comprising distinct embodiments of the present invention, the treatment may comprise the application of radiation, application of physical force (such as ultrasound), or a change in temperature. Other methods known to initiate a chemical reaction are within the scope of the present invention.

In an embodiment, the substrate is substantially inert. Alternatively, the substrate may be a substance that interacts with at least some of the samples of the array.

In an embodiment, individual elements of the array are performance tested for stability upon exposure to at least one external agent. In various embodiments of the present invention, the external agent may comprise a physical force (such as abrasion), electromagnetic radiation (such as UV light), heat, or a chemical reagent.

In an embodiment, the external agent is applied in a constant manner to all samples of the array. Alternatively, the external agent may be applied differentially to at least some of the individual samples in the array.

In an embodiment, spatially resolved measurement tools are used to collect and process data from said array. Preferably, a scanning probe is applied over at least one of the samples. As preferred embodiments, the scanning probe may comprise confocal microscopy, such as Raman confocal microscopy or luminescence confocal microscopy. Alternatively, the scanning probe may comprise two-photon or multiphoton microscopy. In addition, other types of scanning probes known in the art are within the scope of the present invention.

In an embodiment, collecting analytical data from the array is substantially simultaneous for each element of the array. In an alternative embodiment, collecting analytical data from the array is performed separately for each element of the array. Moreover, the methods used for data analysis may include univariate analysis or, in alternate embodiments where multiple parameters are measured, multivariate analysis.

In another aspect, the present invention comprises a method for the generation and performance testing of three dimensional arrays comprising: depositing a plurality of samples onto at least one substrate at discrete and defined positions in a three dimensional format such that each sample is isolated by said substrate from the other samples, and wherein each sample is defined by its (x, y, and z) coordinate to generate a three dimensional array of samples; performance testing the array; collecting data from the performance testing, wherein the data is at least partially defined by its (x, y, and z) coordinate within the sample array; correlating the analytical data collected from the array to the position of samples within the array; and analyzing the analytical data for a parameter of interest.

Preferably, the step of performance testing comprises applying at least one external agent to at least some of the samples of the array. The external agent may comprise a physical force (such as abrasion), electromagnetic radiation (such as UV light), heat, or a chemical reagent. In an embodiment, the external agent is applied in a constant manner to all samples of the array. Alternatively, the external agent may be applied differentially to at least some of the individual samples in the array.

In yet another aspect, the present invention comprises a method for the analysis of three dimensional arrays comprising: applying a scanning probe over a plurality of samples deposited onto at least one substrate at discrete and defined positions in a three dimensional format such that each sample is isolated by said substrate from the other samples, and wherein each sample is defined by its (x, y, and z) coordinate; collecting analytical data from the array, wherein the analytical data is at least partially defined by its (x, y, and z) coordinate within the sample array; correlating the analytical data collected from the array to the position of samples within the array; and analyzing the analytical data for a parameter of interest.

As preferred embodiments, the scanning probe may comprise confocal microscopy, such as Raman confocal microscopy or luminescence confocal microscopy. In an alternate embodiments, the scanning probe may comprise two-photon or multiphoton microscopy. In addition, other types of scanning probes known in the art are within the scope of the present invention.

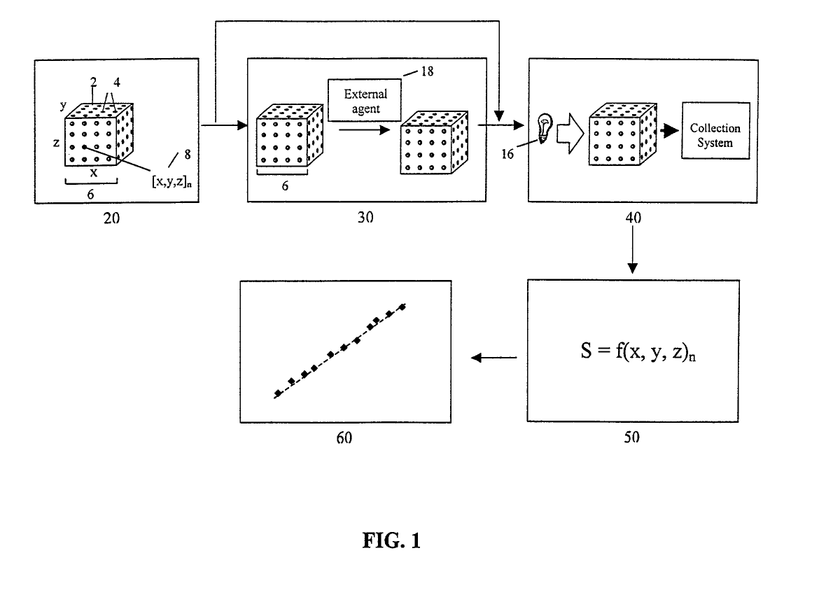

Thus, present invention discloses several embodiments for the fabrication, performance testing, and screening of three dimensional arrays of samples. In one aspect, and referring to FIG. 1, the invention comprises a method for the generation and screening of three dimensional arrays comprising: depositing a plurality of samples (4) onto at least one substrate at discrete and defined positions in a three dimensional format such that each sample is isolated by the substrate (2) from the other samples, and wherein each sample is defined by its (x, y, and z) coordinate 8 to generate a three dimensional array of samples (20); collecting analytical data from the sample array, wherein the analytical data is at least partially defined by its (x, y, and z) coordinate within the sample array (40); correlating the analytical data collected from the array to the position of samples within the array (50); and analyzing the analytical data for a parameter of interest (60). In an embodiment, the method includes performance testing of the array (30), generally by application of an external agent 18 to samples in the array.

Thus, the samples in the array, and data derived from the array is defined in part by the x, y, and z coordinates of each sample. As defined herein, x, y, and z describe the three different directions which characterize three dimensional space as in understood in conventional mathematical terminology. Thus, x describes one direction which is perpendicular to both y and z and on the same plane as both individually, but not simultaneously; y describes a direction which is perpendicular to both z and z and on the same plane as both individually, but not simultaneously, and z describes a direction perpendicular to y and x, and on the same plane as both individually, but not together.

Three dimensional arrays generated by stacking microtiter plates have been described. In this array format, the microtiter plate wells are interconnected by channels for selective routing of reagents (U.S. Pat. No. 6,083,682 and U.S. Pat. No. 6,168,914 B 1). In contrast, the arrays of the present invention comprise a collection of samples which are not interconnected, but are physically and chemically isolated from each other by the array substrate. Thus, the elements of the arrays of the present invention are substantially solid and do not coalesce or mix with neighbor elements over the time period of the combinatorial experiment. Instead, the arrays of the present invention are substantially solid-state units which are suitable for simultaneous performance testing and/or screening of array samples.

The methods of the present invention are particularly well-suited to the analysis of combinatorial libraries, but may be applied to other formats requiring screening of multiple samples as well. Samples may be made in a separate environment and then loaded onto the array for testing, or may be generated after deposition of starting reagents in an array format. It is envisioned that arrays may be sized to accompany macroscopic samples (i.e. samples ranging in size from femtoliters up to milliliters), or may be very small, as for example, three dimensional biochips, and the like.

The method of the present invention may be used to analyze a parameter of interest in a sample (or a library of samples), wherein a parameter of interest comprises a chemical, physical, or mechanical aspect of the sample which can be monitored experimentally. Parameters of interest include, but are not limited to, starting reaction components, chemical intermediates, reaction by-products, final products, and mechanical parameters such as moduli, and the like. For example, in the synthesis of melt polycarbonate, a parameter of interest may be the starting components bisphenol A or diphenyl carbonate, oligomer intermediates, phenol, Fries products, or the final polymer. Reference will now be made in detail to embodiments of the present invention, examples of which are illustrated in the accompanying drawings.

Fabrication of 3D Arrays

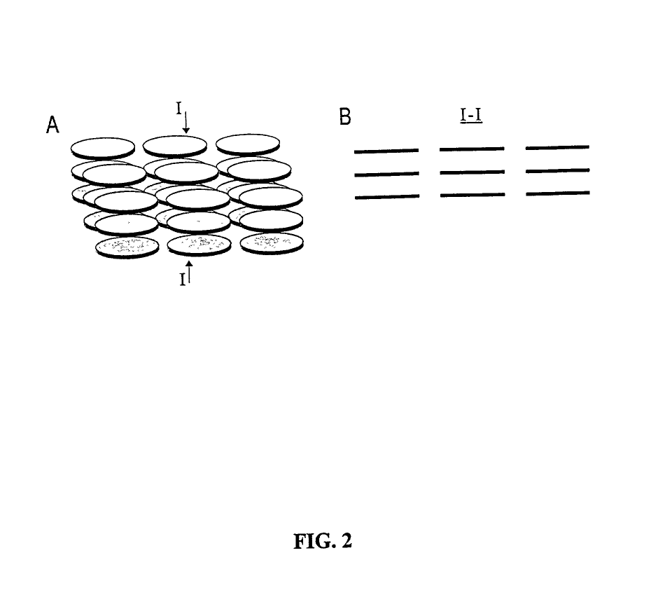

FIG. 2 presents an array in which a set of two dimensional (2D) libraries are assembled to form a three dimensional structure. Examples of two-dimensional structures which may be assembled in a three dimensional format include biochips, micro-titer plate reactors, triangular arrays, and the like. Unlike the 2D arrays used previously, however, arrays of the present invention comprising multiple two dimensional structures are assembled in such a way that the resulting three dimensional array comprises a unitary structure suitable for simultaneous testing of all the member elements.

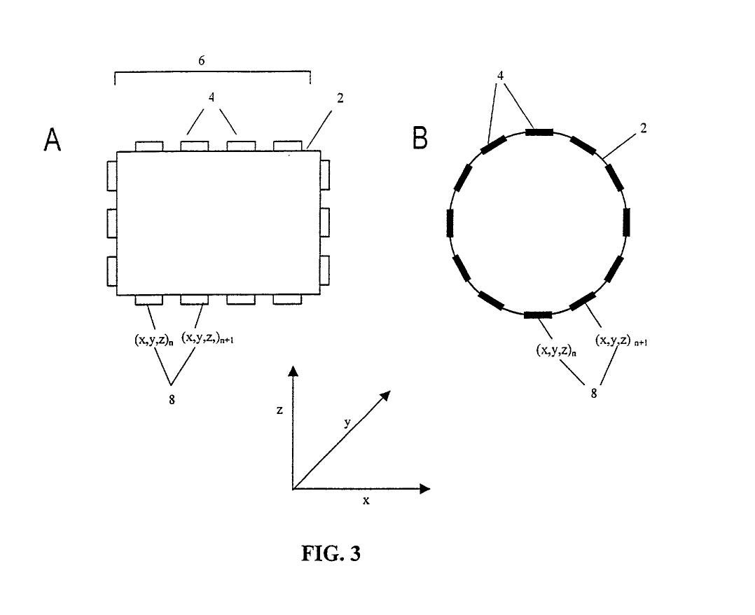

In another embodiment, and referring now to FIG. 3, the library of individual samples 4 is deposited on an outer surface of a three dimensional (3D) substrate 2 to generate a three-dimensional array 6 such that each sample is isolated by the substrate from the other samples, and wherein each sample is defined by its (x, y, and z) coordinate 8 to generate a three dimensional array of samples. For example, it can be seen that neighboring samples will have coordinates which vary by at least one x, y, or z coordinate, and that the (x, y and z) coordinate 8 identifies the particular sample 4 deposited onto substrate 2.

Deposition can be accomplished using known deposition methods such as evaporation, laser ablation, spraying, and the like. Such deposition techniques may be combined with discrete or gradient masking techniques known to those skilled in the art. Additionally, variation among elements or regions of the 3D library can be induced using known non-masking methods. For example, different regions of a 3D substrate may be dip-coated with variable formulations. Alternatively, other parameters for coating may be varied (e.g. thickness, curing conditions, underlying substrate).

Substrates suitable for the arrays of the present invention include conductive materials such as carbon, conductinve polymers (such as polypyrrole) and metals (e.g. zinc, copper) semi-conductive materials (e.g. silicon, gallium arsenide), and nonconductive materials such as polymers (e.g. polypropylene, polyethylene), alumina, quartz and glass.

For example, thin-film deposition techniques in combination with physical masking techniques or photolithographic techniques can be used to apply samples onto a substrate surface and thereby create the arrays of the present invention. See U.S. patent application Ser. No. 90/681,432 to Potyrailo, incorporated by reference herein, for a description of thin film deposition techniques used to generate one dimensional arrays. Deposition techniques can generally be broken down into the following four categories: evaporative methods, glow discharge processes, gas-phase chemical processes, and liquid-phase chemical techniques. Included within these categories are, for example, sputtering techniques, spraying techniques, laser ablation techniques, electron beam or thermal evaporation techniques, ion implantation or doping techniques, chemical vapor deposition techniques, as well as other techniques used in the fabrication of integrated circuits. All of these techniques can be applied to deposit highly uniform layers of samples onto a substrate. Moreover, by adjusting the relative geometries of the masks, the delivery source and/or the substrate, such thin-film deposition techniques can be used to generate uniform gradients at each reaction region on the substrate or, alternatively, over all of the reaction regions on the substrate. For an overview of the various thin-film deposition techniques which can be used in the methods of the present invention, see, for example, Ballantine, D. S., Jr., et al., In Acoustic Wave Sensors: Theory, Design, and Physico-Chemical Applications, 1997, Chapter 6, Academic Press: San Diego, Calif., incorporated herein by reference for all purposes.

In one embodiment, library samples can be deposited onto the substrate of the array using evaporative methods preferably in combination with physical masking techniques (e.g. Lundstrom, I., et al., Nature, 1991, 352: 47-50) and using gradient deposition methods (e.g. Hanak, J. J., et al., J. Vac. Sci. Technol., 1971, 8: 172-175). Generally, in thermal evaporation or vacuum evaporation methods, the following sequential steps take place: (1) a vapor is generated by boiling or subliming a target material; (2) the vapor is transported from the source to a substrate; and (3) the vapor is condensed to a solid film on the substrate surface. Evaporants, i.e., target materials, which can be used in the evaporative methods cover a large range of chemical reactivities and vapor pressures and, thus, a wide variety of sources can be used to vaporize the target materials. Such sources include, for example, resistance-heated filaments, electron beams, crucible-heated-by-conduction, radiation or rf-inductions, as well as arcs, exploding wires and lasers. In preferred embodiments of the present invention, thin-film deposition using evaporative methods is carried out using lasers, filaments, electron beams or ion beams as the source. Successive rounds of deposition, using evaporative methods can be used to deposit library samples onto a substrate to generate the three dimensional arrays of the present invention.

In another embodiment, Molecular Beam Epitaxy (MBE) is used to grow epitaxial thin-films. In this method, the films are formed on single-crystal substrates by slowly evaporating the elemental or molecular constituents of the film from separate Knudsen effusion source cells (deep crucibles in furnaces with cooled shrouds) onto substrates held at temperatures appropriate for chemical reaction, epitaxy and re-evaporation of excess reactants. The Knudsen effusion source cells produce atomic or molecular beams of relatively small diameter which are directed at the heated substrate, usually silicon or gallium arsenide. Fast shutters are interposed between the source cells and the substrates. By controlling these shutters, superlattices with precisely controlled uniformity, lattice match, composition, dopant concentrations, thickness and interfaces down to the level of atomic layers may be generated.

In addition to evaporative methods, samples can be deposited onto the substrate using glow-discharge processes preferably in combination with physical masking techniques. The most basic and well known of these processes is sputtering, i.e., the ejection of surface atoms from an electrode surface by momentum transfer from bombarding ions to surface atoms. Sputtering or sputter-deposition is a term used by those of skill in the art to cover a variety of processes, all of which can be used in the methods of the present invention. One such process is RF/DC Glow Discharge Plasma Sputtering. In this process, a plasma of energized ions is created by applying a high RF or DC voltage between a cathode and an anode. The energy ions from the plasma bombard the target and eject atoms which are then deposited on a substrate, a sensor layer. Ion-Beam Sputtering is another example of a sputtering process which can be used to deposit thin-films of the various barrier coating materials on a substrate. Ion-Beam Sputtering is similar to Glow Discharge Plasma Sputtering except the ions are supplied by an ion source and not a plasma. It will be apparent to one of skill in the art that other sputtering techniques (e.g., diode sputtering and reactive sputtering) and other glow-discharge processes can be used in the methods of the present invention to deposit samples onto a substrate. Successive rounds of deposition, through different physical masks, using sputtering or other glow-discharge techniques, can be used to deposit samples onto a substrate to generate three dimensional arrays of the present invention.

Alternatively, the various sample materials can be deposited onto the substrate using Chemical Vapor Deposition (CVD) techniques preferably in combination with physical masking techniques. CVD involves the formation of stable solids by decomposition of gaseous chemicals using heat, plasma, ultraviolet, or other energy sources, or a combination of energy sources. Photo-Enhanced CVD, based on activation of the reactants in the gas or vapor phase by electromagnetic radiation, usually short-wave ultraviolet radiation, and Plasma-Enhanced CVD, based on activation of the reactants in the gas or vapor phase using a plasma, are two particularly useful chemical vapor deposition techniques. Successive rounds of deposition, through different physical masks, using CVD technique can be used to deposit samples onto a substrate to generate three dimensional arrays of the present invention.

Various samples of interest can also be deposited onto the substrate using a number of different mechanical techniques preferably in combination with physical masking techniques. Such mechanical techniques include, for example, spraying, spinning, dipping, draining, flow coating, roller coating, pressure-curtain coating, brushing, etc. Of these, the spray-on and spin-on techniques are particularly useful. Sprayers which can be used to deposit thin-films include, for example, ultrasonic nozzle sprayers, air atomizing nozzle sprayers and atomizing nozzle sprayer. In ultrasonic sprayers, disc-shaped ceramic piezoelectric transducers convert electrical energy into mechanical energy. The transducers receive electrical input in the form of a high-frequency signal from a power supply that acts as a combination oscillator/amplifier. In air atomizing sprayers, the nozzles intermix air and liquid streams to produce a completely atomized spray. In atomizing sprayers, the nozzles use the energy from a pressurized liquid to atomize the liquid and, in turn, produce a spray. Successive rounds of deposition, through different physical masks, using mechanical techniques, such as spraying, can be used to deposit samples onto a substrate.

Samples may be applied in a variety of thickness ranges. Thus, the individual samples of the array suitably have a thickness from 0.1 nm to 1 cm, more preferably from 1 nm to 1 mm, and more particularly from 10 nm to 100 micrometers.

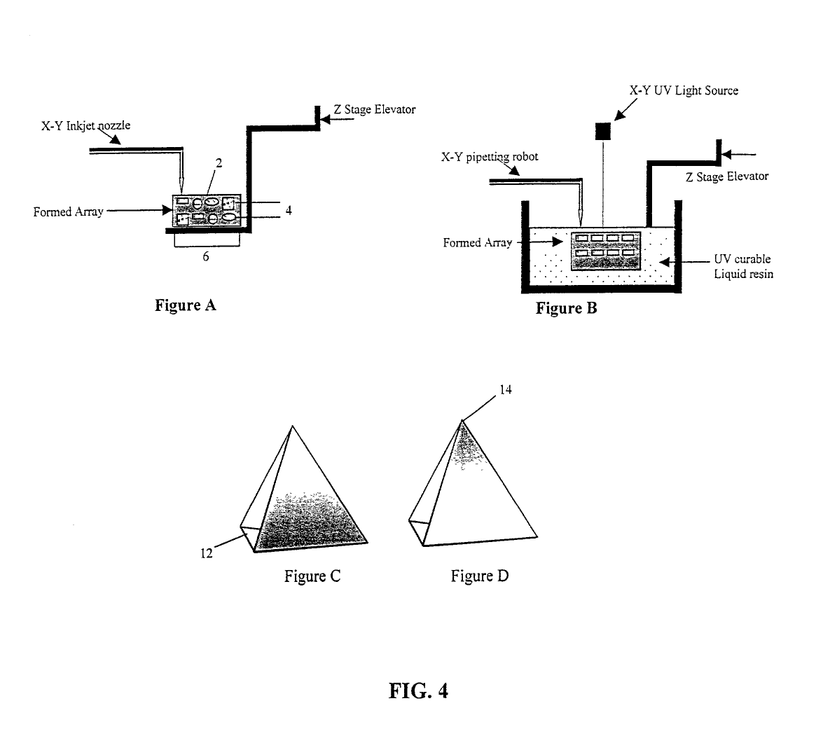

In another embodiment, and referring now to FIG. 4A, a three dimensional library 6 is produced by inducing a spatial variation within a 3D structure. Such an array 6 may be generated by adding samples 4 into a preformed three dimensional substrate 2 such as wax or a polymer. Using an inkjet or other applicator, samples of different composition and/or shape may be distributed in the substrate. In an embodiment, the samples deposited in the array comprise final reaction products. In an alternate embodiment, the samples deposited in the array comprise an incomplete set of reagents required for the reaction of interest and the final library (or array) is generated by adding a small volume of the missing component to each sample address and allowing the reaction to proceed.

Alternatively, and referring now to FIG. 4B, samples can be deposited in a three dimensional arrangement, and the substrate formed around the samples. For example, a stereolithography apparatus can be used to construct a three dimensional substrate by scanning a UV light source on a thin film of photopolymerizable material. Examples of such a substrate are acrylate-based monomers that upon a radical photopolymerization exhibit a liquid-solid phototransformation (Bertsch, P., 2000, et al., Rapid Prototyping J., 6: 259-266). Other materials suitable for photopolymerization are described in U.S. Pat. No. 6,200,737 and WO/0013004 and include the following monomers, polymers and copolymer compositions and their derivatives: polyethylene glycol, polycaprolactone, polyarylamide, methyl methacrylate [MMA], 2-hydroxyethyl methacrylate, siloxane, dimethylsiloxane, acrlyamide, methylenebisacrylamide BA), poly (1,4-butylene) adipate, poly (2,6-dimethyl-1,4-phenyleneoxide) [PDPO], triethoxysilyl-modified polybutadiene (50% in toluene) [PS078.5], diethoxymethylsilyl-modified polybutaidiene in toluene [PS078.81], acryloxypropylmethyl-cyclosiloxane [CPS2067], (80-85%) dimethyl-(15-20%) (acryioxypropyl) methylsiloxane copolymer [PS802], poly(acryloxypropylmethyl)siloxane [PS901.5], (97-98%) dimethyl-(2-3%) (methacryloxypropyl)methylsiloxane copolymer [PS851], poly(acrylonitrilebutadiene-styrene) [PABS], poly(methyl methacrylate), poly(styrene-acrylonitrile 75:25) [PSAN], acryloxypropylmethylsiloxane-dimethylsiloxane copolymer, methylstyrenes, styrenes, acrylic polymers, methylstyrene divinyl benzene, metallocene-based ethylenes, ethylene oxides, aromatics, alkyl or alkoxy substituted aromatics, dimethyle siloxanes, alkythiophenes, pyrroles, thiophenes, 3-methyl thiophene, 3,4-dimethyl thiophene, anilines, N-vinyl carbozole, p-wphenylene vinylene, acetylenes, styrenes, p-phenylenes, ethylenes, propylenes, dienes, vinyl chlorides, carbonates, vinyl acetates, urethanes, butadienes, acrylates, methacrylates, chloromethylated styrenes, vinylamines, and arsenic pentafluoride or iodine-doped combinatorial polymers comprising polyacetylene, poly-p-phenylene, polypyrrole and polyphenylene sulfide, polyimides, polycarbonates, phtha locya nines, styrene-butadiene-styrene copolymers, cyclophanes, phthalocyanines, thiols, silanes, lipids, nucleic acids, enzymes, antibodies, triethanolamine, quadrol, ethylene dinitrotetraethanol, ascorbic acid, capiscum, L-glutamic acid, pyridoxine, triphenylamine, methyl-trioctylphosphonium dimethyl phosphate, glutathione, NAD, ethylene maleate, triethanolamine, vinyl stearates, collodion, butadiene-acrylonitriles, phenyl phenol, hydroxy terminated polybutadiene, vinyl isobutyl ethers, polyvinyl chlorides, caprolactones, caprolactone triols, butadiene methacrylate, methyl methacrylates, polystyrenes, ethylene glycol methyl ether, vinyl carbozoles, abietic acid, octadecyl vinyl ether/maleic anhydride, polyethylenes, ethylcellulose, fluoropolyols, siloxanes, alkylaminopyridyl-substituted polysiloxanes, poly(4-vinyl hexafluorocumyl alcohol), hexafluor-2-propanol-substtuted polysiloxanes, polyepichlorohydrin, polybis(cyanopropyl)-siloxanes, polyvinyl tetradecanals, polyisobutylenes, poly(trifluorpropyl)methylsiloxanes, polyethylene maleates, polyvinyl propionates, polyethyl enimines, polyphenyl ethers, docosanol, diglycerols, polydiphenoxyphosphazenes, diethyleneglycol adipate, polychloroprenes, acrylonitrile butadienes, apiezon L, biscyanoallyl polysiloxane, polyepichlorohydrin, pheynimethyidiphenyisilocone, vinyl-modified tyifluoropropylmethylsilicone, tributoxyethyl phosphate, poly(hexyl arylate), poly(2-hydroxyethyl arylate), W-ethyl o,p-toluene sulfonamide, and phenyl ethers.

Photoinitiators which may be used include: (A) metals such as Tris (2,2¹-bipyridyl) Ruthenium (II) chloride & lsqb;Ru(bpy) 3²⁺], and pentaaminechlorocobalt (III) chloride & lsqb;Co (NH3)5 Cl²⁺] initiating system; (B) radical moieties such as benzoin ethyl ether, benzophenone, benzoyl peroxide, AIBN, 2-2¹ azobis(butyronitrile), 2,2¹-dimethoxy-2-phenylacetophenone; 2,2¹-diethoxyacetophonone; and (C) others such as anthraquinone in combination with tetrohydrofuran, ribofavin in combination with ammonium persulfate, and phloxine B in combination with triethylamine.

Photocrosslinkable chemicals entities that may be employed to generate photopolymerizable three-dimensional arrays include: (A) monomers such as acrylamide, N,N-methylene bis(acrylamide), hydroxyethylmethacrylate, styrene, vinyl acetate, N-(3-aminopropyl)meth-acrylamide hydrochloride, methyl methacrylate, N-vinlyl pyrrolidone, acryloxysuccinimide, pyrrole, aniline, and thiophene; (B) comonomers with dimethylsiloxane: (acryloypropyl) methyl (15-20%), (aminopropyl)methyl (3-5%), and (methacryloxypropyl)methyl (2-3%); (C) T-structure polydimethlysiloxanes: methacryloxypropyl (25-50%) and vinyl (50-75%); and (D) others such as hydrogels (polyacrylamide, polyvinyl alcohol, poly(2-hydroxyethyl methacrylate), poly-N-(2-hydroxypropyl methacrylamide, poly-vinyl pyrrolidone, polymethyl methacrylate (as adjuvants)) hydrophobic compounds (ethylene vinyl acetate copolymer, silicone elastomers, microporuos polypropylene, cross-linked (meth)acrylates); blends (latex, polysaccahride-glycolide, acrylates); and others such as polylactic acid (polylactide), polyglycolic acid (polyglycolide), poly(c-carprolactone): polyvalerolactone, poly(hydroxybutyric acid-co-hydrovaleric acid), poly othro esters, poly alkylcyanoacrylates, synthetic polypeptides, cross-linked polypeptides and proteins, natural polymers (albumin, gelatin, starch), polyanhydrides, monomers for sebacic acid, bis(p-carboxy-phenoxy)-propane, dodecandedioic acid, cellulose acetate trimelltiate, hydroxypropyl methyl cellulose phthalste, cellulose acetate, cellulose acetate propionate, cellulose triacetate, copolymers of methacrylic acid and methacrylic acid methyl ester, Eudragit L100.

Solvents include ethanol, methanol, propanol, dichloromethane, tetrahydrofuran (THF), dimethyl sulfoxide (DMSO), dimethyl formamide (DMF), ethyl acetate, ethylene glycol, and water.

The apparatus can be modified by the addition of a robotic pipet which adds small quantities of chemical substances to distinct regions of a film of non-polymerized photopolymer (FIG. 4B). After sufficient time for the substance to mix in the photopolymer by diffusion (i.e. to create a array elements having the appropriate size), the photopolymer film is polymerized by application of UV light. The structure is then lowered by the thickness of the film using a Z-stage elevator and the process repeated, thereby building up a three dimensional structure with thin disc-shaped regions containing different chemical substances. In an embodiment, the samples deposited in the array comprise final reaction products. In an alternate embodiment, the photopolymerizable material comprises a first reaction component that interacts with the reaction components deposited using the robotics pipet.

Alternatively, and referring now to FIGS. 4C and 4D, the array may be formed as a tetrahedron. In an embodiment, the array is formed by diffusing materials inward from the faces of the tetrahedron. Thus, FIG. 4C depicts diffusion of one component from a plane 12 of a tetrahedron. In an embodiment, the array is formed by diffusing materials inward from the vertices of the tetrahedron, as shown in FIG. 4D, which depicts diffusion of one component from 2 vertex 14 of a tetrahedron. The tetrahedron is uniquely suited to the study of materials comprising four components (or four components of interest to be measured) because it is the geometrical embodiment of a four-factor simplex. Thus, in a simplex system, factors are constrained such that the sum of the factors equals a constant. Preferably, the sum of the factors will equal 1.00 (or 100%). The simplex can be represented as a regular sided figure with k+1 vertices in k dimensions. For k=2, the simplex design is an equilateral triangle. For k=3, it is a regular tetrahedron.

Other methods of applying multiple samples known in the art may be used to create the three dimensional arrays of the present invention. For example, in an embodiment, the samples are deposited using a multichannel pipet, where each channel comprises unique samples or reaction components. In yet another embodiment, the samples are deposited in the substrate using a two-dimensional (x-y matrix) applicator such as the applicators used in commercially available microarrayer systems (Affymetrix Modes 417 and 427; Affymetrix Inc., Santa Clara, Calif.).

In addition to the specific embodiments described above, methods known in the art of rapid sequential or parallel material deposition are well-suited to generating arrays of the present invention. There are three general classes of methods for three dimensional structuring; these classes can be described as additive, subtractive or formative (see e.g. J.-M. Breguet and A. Bergander, 2001, Proc. SPIE Vol: 4568, paper 30, in press; Karapatis, N. P., et al., Rapid Prototyping J., 4: 77-89, 1998). Using these methods, three dimensional objects can be made by fusing individual particles (additive), by removing material from a solid block (subtractive); or by pressing on opposite sides of a structure to change its shape without adding or removing material (formative). In addition, combinations of the processes (additive, subtractive and formative) are also possible.

For example, microstereolithography is an additive fabrication method that allows building small-sized (micro) three dimensional structures which may comprise complex shapes. Microstereolithography has evolved from the rapid prototyping industry, and is based on a layer-by-layer induced polymerization of a liquid resin. Objects made of a few hundred to several thousands of layers can be built with a spatial resolution of several microns in three directions (Bertsch, P., et al., 2000, Rapid Prototyping J., 6(4): 259-266).

Another additive method is Selective Laser Sintering (SLS) (D. T. Pham and R. S. Gault, 1998, Intern. J. of Machine Tools and Manufacture, 38 (10-11): 1257-1287). In SLS a layer of powder is deposited on a support and compacted by a rolling device. A computer-controlled laser beam scans a two-dimensional cross-section of the part, selectively sintering the layer. A new layer is then deposited, compacted and sintered. After completion of the part, the unfused powder is removed. This method allows sintering of polymers, metal or ceramic powders. It is also possible to locally introduce variation of material composition.

An alternative to SLS is the Laser Generating (LG) method (see e.g., Karapatis, N. P., et al., 1998, Rapid Prototyping J., 4:77-89). In LG, the material is not deposited prior to the laser treatment, but is gas delivered directly to the focal zone of the laser beam. The laser scans a two-dimensional cross-section of the part, and the part is then built layer-by-layer as in SLS. Compared to SLS, LG allows a higher density of the material delivered to the part, such that the deposited material is less porous. Although LG allows for controlled delivery of the material of interest to the unit, the LG method requires a high power laser and high quality laser beam.

Electron Beam Induced Deposition (EBID) is an alternative to SLS and LG (Utke, B., et al., June 2000, Microelectronic Engineering, 53(1-4): 261-264). Considered more accurate than either SLS or LG, EBID uses a high-intensity electron beam within an electron microscope to induce the formation of structures on the scanned surface. High aspect ratio structures with lateral resolution a several tens of nanometers can be obtained.

Another additive fabrication method, 3-Dimensional Printing (3DP), comprises a three dimensional extension of the inkjet printing devices (Karapatis, N. P., et al., 1998, Rapid Prototyping J., 4:77-89). In 3DP, a three dimensional structure is built layer-by-layer using a jet printer to apply a binder material on a thin powder layer. The unbounded powder is then removed and a new layer processed. 3DP offers the advantages of rapid fabrication of three dimensional structures at a relatively low cost. Limitations of the method include the relatively low resolution of sample deposition (about 0.5 mm) and the high porosity of the finished part. The materials generally employed by 3DP are metal or ceramic powders, or metal-ceramic composites.

One subtractive method suitable for generating arrays of the present invention is the Electro Discharge Machining (EDM) (e.g., Langen, H., et al., 1998, International J. of Electrical Machining, 3: 65-69). EDM is a well-established industrial technology used to produce manufacturing tools as well as small, but very accurate parts made of conductive and semi-conductive materials. A combination of Electro Discharge Machining and Electrochemical Etching (ECDM) allows machining of non-conductive materials such as glass or ceramics. The ECDM provides micrometer accuracy of material deposition and surface quality suitable for optical applications.

Performance Testing of 3D Arrays

Generally performance testing of three dimensional arrays is performed under conditions that simulate factors (e.g. chemical degradation, UV exposure, weathering) to which the materials of interest may be exposed. Because of the small sample size, the large number of samples (and variables) which can be tested, and the ability to control the level of exposure to the agent of interest, combinatorial libraries provide information regarding the stability of many more materials of interest compared to conventional in situ (field) testing procedures.

Performance testing may be performed using targeted (spatially resolved) or non-targeted test conditions. Thus, in an embodiment, test conditions are constant for every element (or region) of the 3D array. Constant test conditions include, but are not limited to, physical conditions (e.g., gravity, temperature, pressure, electromagnetic radiation, and the like), mechanical conditions (e.g., abrasion tests over the surface of a library), or chemical (e.g., application of a liquid or gas chemical which penetrates through the array).

For example, in an embodiment, an array of samples deposited on the surface of a three dimensional structure may be tested for resistance to abrasion. Standardized abrasion tests known in the art may be used with the arrays of the present invention. Such standardized abrasion tests include, but are not limited to, the ASTM D968 Standard Test Method for Abrasion Resistance of Organic Coatings by Falling Abrasive, the ASTM D4060 Standard Test Method for Abrasion Resistance of Organic Coatings by the Taber Abraser, the ASTM F735 Standard Test Method for Abrasion Resistance of Transparent Plastics and Coating Using the Oscillating Sand Method, the ASTM D1044 Standard Test Method for Abrasion Resistance of Transparent Plastics to Surface Abrasion, and the ASTM D1242 Standard Test Method for Resistance of Plastic Materials to Abrasion.

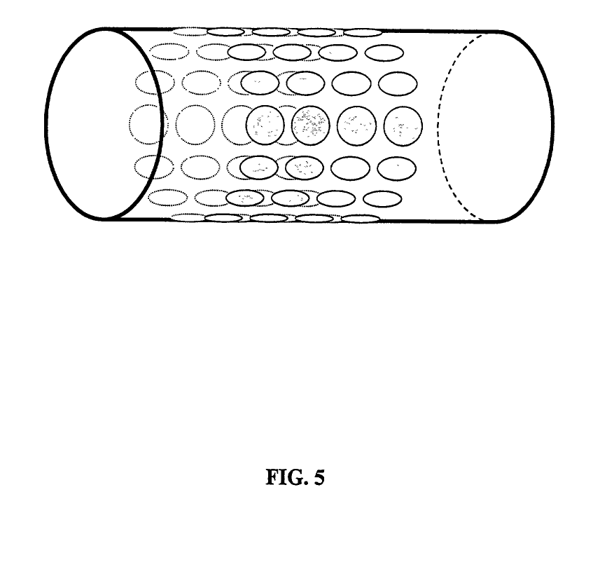

Samples deposited on the surface of a three dimensional structure may also be performance tested for their resistance to weathering. For example, a significant factor in the lifetime of polymers to outdoor weathering is the ultraviolet radiation (UV dosage) received by a given sample. On a macroscopic scale, outdoor weathering tests are usually performed in environment in which UV exposure is high (e.g. Florida, Arizona), and are conducted over a period of from one to ten years. In contrast, combinatorial libraries allow for rapid testing of large numbers of small-scale samples using controlled doses of UV radiation. For example, an array of polymers may be generated on the surface of a three dimensional structure as shown in FIGS. 3 and 5. An ASTM G-26 xenon arc lamp may be used to apply UV radiation locally to each sample on the array, with degradation of organic materials manifested by a loss of gloss and/or a yellowing in color, especially for pigmented materials. For example, a widely used analytical technique for determination of UV degradation involves measurements of the Yellowness Index (YI) according to ASTM procedure D-1925, wherein a smaller change in YI over the testing period suggests a higher photo-stability of a sample, and better resistance to weathering. Thus, screening of three dimensional combinatorial arrays allows for a significant reduction in the overall time required for testing polymers for weathering resistance.

Samples within a three dimensional array may also be performance tested for stability to agents that can permeate the array such that samples within the array are exposed to the agent/reagent of interest. For example, samples may be performance tested for their resistance to chemical degradation. Materials which may be of interest to test for chemical stability include various polymeric materials which have been synthesized to be resistant to chemical degradation. For example, combinatorial libraries tested for resistance to chemical reagents include, but are not limited to, polymers (such as polycarbonate and the like), polymer blends (such as polycarbonate/polystryrene blends), silicones, copolymers (such as polycarbonate/polyorganosiloxane copolymers), polyetherimide resins, and many others. Chemical reagents such as, but not limited to, fuels, alkaline and acidic solutions, water, organic solvents (both polar and non-polar) and solvent mixtures, may be used to screen samples for chemical stability. Generally, samples are tested for resistance to a chemical reagent by exposing the sample to the reagent of interest for known periods of time. In many cases, conventional methods of measurement of chemical resistance require exposure of a sample to a reagent for extended periods of time (i.e. typically 7 days at room temperature or 3-4 days at elevated temperatures). Thus, conventional methods are generally overly time-consuming and/or resource-consuming for testing the numbers of test samples that are generated using combinatorial chemistry. Moreover, traditional measurement techniques are difficult to apply for measurements of multiple small samples simultaneously.

In an embodiment, samples are deposited on the surface of a three * dimensional array (FIG. 5), and the array exposed to a chemical reagent. The samples may then be tested for resistance to the reagent. For example, chemical degradation may be monitored by detecting an increase in degradation products or a loss in sample. Methods to monitor sample degradation include, but are not limited to, spectroscopic analysis, mass spectrometry, separation techniques (such as chromatography), gravimetric analysis, and other analysis techniques known in the art.

Alternatively, the samples may be subjected to a physical performance test after exposure to the reagent of interest, and the results compared to non-treated samples. For example, using the ASTM F735 Standard Test Method for Abrasion Resistance of Transparent Plastics and Coating Using the Oscillating Sand Method, a three dimensional array is exposed to the test (abrasion) after exposure to a solvent for a predetermined time.

In another embodiment, samples deposited within a three dimensional substrate (e.g. FIGS. 4A and 4B) may be subjected to an environmental exposure, as for example an elevated temperature which is constant across all elements of the array. For example, samples deposited on the surface of a three dimensional structure or within a three dimensional array structure may also be performance tested for their hydrolytic stability. Conventional techniques for measuring the hydrolytic stability of polymers typically involves autoclaving the sample and then dissolving the sample for molecular weight determination by gel permeation chromatography (GPC). There are, however, significant drawbacks associated with the conventional approach. The method is destructive in that it requires the sample be dissolved, and is therefore inappropriate for field (in situ) studies of polymer integrity. Also, the method is time-consuming and generates non-biodegradable waste.

Thus, in an embodiment, and referring again to the array prototype of FIG. 5, polymers arranged on the surface of a three dimensional substrate may be subjected to high temperature, high pressure aqueous conditions (e.g. autoclaving) to promote chain hydrolysis. The samples may then be performance tested for the extent of hydrolysis by measuring fluorescence associated with hydrolytic degradation (Potyrailo et al., U.S. patent application Ser. No. 01/31,780). Alternatively, samples deposited on the surface of a three dimensional structure may also be performance tested for “hazing” and ductility. Hazing refers to the tendency of worn polycarbonate samples to become refractive to visual light and is typically evaluated by measuring light transmittance through a sample (e.g. U.S. Pat. No. 4,436,879).

In an embodiment, test conditions are identical for each sample, and it is the sample composition that varies. For example, in an embodiment, a library of samples is deposited as a three dimensional array, the array is subjected to autoclaving, and fluorescence of each sample monitored. An array which could be used for this type of experiment is shown as FIG. 5. In another embodiment, test conditions are variable for every element of an array (i.e. changing the dose of UV light depending upon sample address). An array which could be used for this type of experiment is shown in FIG. 4, wherein the radiation dosage at different z-positions in the array would vary depending upon the z-position of the sample. Thus, variable conditions include, but are not limited to, physical conditions (e.g. temperature gradients or light), mechanical conditions (e.g. abrasion or stress tests which may vary depending on the sample position or geometry), or chemical (e.g. application of a chemical or gas as a gradient throughout the array).

Screening of 3D Arrays

Screening of 3D arrays may be performed using spatially resolved measurement tools. In an embodiment, each sample of a larger array is analyzed individually. Alternatively, samples comprising a multi-element array may be processed simultaneously. Tools for screening 3D arrays include confocal or other types of microscopy, video recording with varying depth of resolution, optical time domain techniques, and other known methods.

For example, an entire multi-element array can be analyzed using imaging techniques such as, but not limited to: (a) the analysis of scattered, reflected, or transmitted light from samples irradiated with light from a light source; (b) the analysis of radiation emission from samples upon excitation with a source of electromagnetic or ionizing radiation; and (c) the analysis of thermal or luminescence radiation emitted from samples during a chemical reaction, mechanical testing, and the like. The array can be analyzed using sensor array techniques where each sample in the array is analyzed with an individual sensor or a sensor array and the data is collected from the sensors simultaneously.

For example, confocal microscopy is particularly well-suited for analyzing three dimensional arrays. With a confocal optical set-up it is possible to detect regular scattered light and also other kinds of secondary radiation emerging from the laser focus. Examples are fluorescence, different types of photoluminescence, and Raman scattering. In this way the instrument operates as a confocal luminescence or Raman microscope.

Thus, in a confocal microscope, parallel laser light is focused to a diffraction-limited spot on the sample with a microscope objective of high numerical aperture. Light scattered from this spot in backward direction is collected and collimated by the same objective and focused through a pinhole onto a photodetector. Only light from the focus can penetrate through the pinhole; light from other depths in the sample is efficiently blocked. As a consequence, out-of-focus regions do not contribute to the signal. This provides the ability to investigate different depth layers separately and to obtain three dimensional microscope images.

Thus, with normal fluorescence microscopy, three dimensional emissions cannot be resolved because of light emitted and scattered by regions of the sample which are not in focus. In confocal fluorescence microscopy, however, because small apertures are placed in the light path at points which are confocal to the focal point within the sample, almost all of the out-of-focus light is blocked, thereby allowing detection of fluorescence from the point of interest. By scanning the light source (i.e. a laser beam) across the sample in the X and Y directions, a high-resolution image of a thin slice of the sample is constructed. A series of such optical slices through the thickness of the sample (i.e. in the Z direction) allows for the generation of a three dimensional sample. The spot focused on the center of the pinhole is the “confocal spot.” Raster scanning comprises the use of a laser (or other excitation device) to scan across the sample on a specific X-Y plane and illuminate a specific area of the sample.

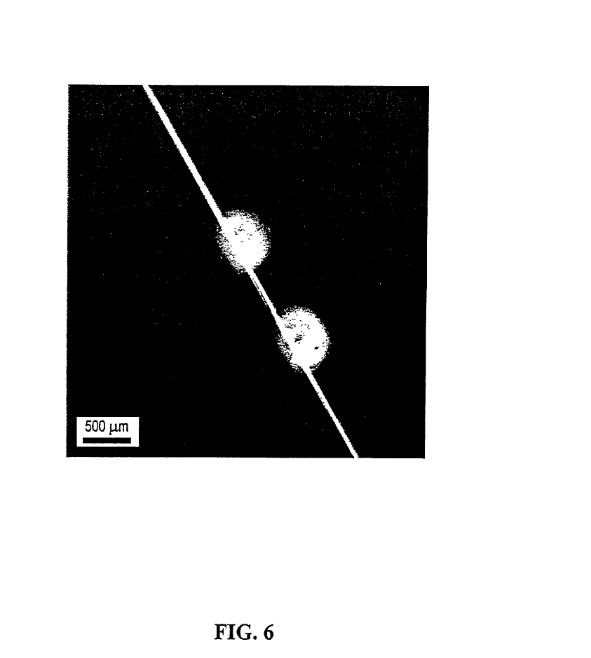

For example, and referring now to FIGS. 6-8, laser scanning confocal microscopy may be used to analyze the three dimensional arrays of the present invention. Laser scanning confocal microscopy provides a three dimensional reconstruction of stacks of two dimensional images.

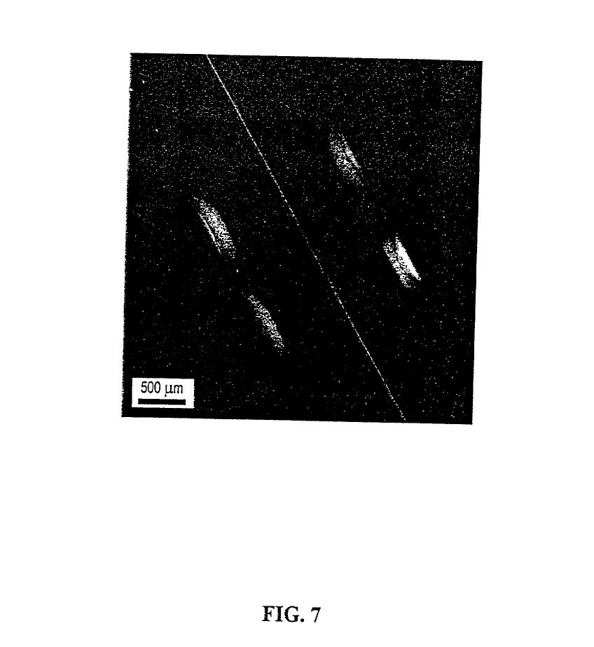

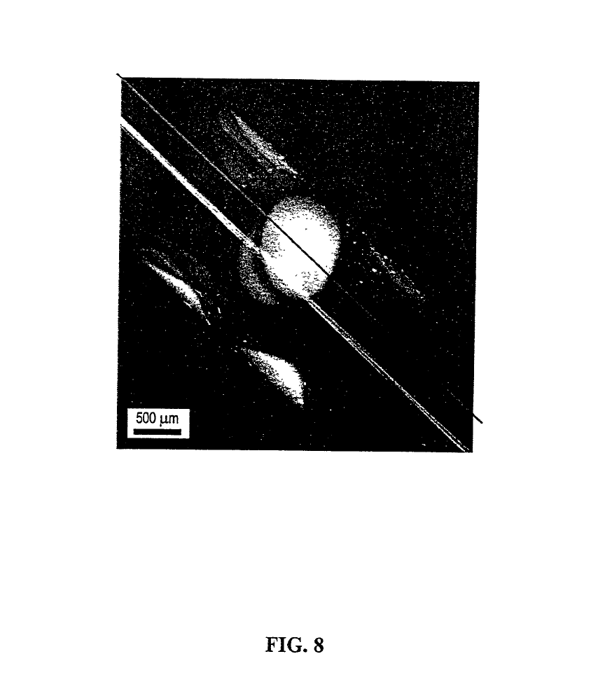

Thus, in an embodiment, a two-dimensional image (referred to as an optical section) of a small partial volume of the specimen which is centered around the focal plane is generated by performing a raster sweep of the specimen at that focal plane. As the laser scans across the specimen, the analog light signal is detected by the photomultiplier is converted into a digital signal, and that digital signal added to a pixel-based image displayed on a computer monitor. The relative intensity of the fluorescent light emitted from the certain region of the sample corresponds to the intensity of the resulting pixel in the image (e.g. FIGS. 6 and 7). The plane of focus (i.e. the XY plane) is selected by a computer-controlled fine-stepping motor which moves the microscope stage up and down. Typical focus motors can adjust the focal plane in increments of 0.1 micron or less. A three dimensional reconstruction of a specimen is generated by stacking two-dimensional optical sections collected in series (FIG. 8). Commercially available confocal microscopes such as those produced by Nikon, Olympus, Renishaw, (Renishaw Ramascope 2000; Renishaw, Inc., Hoffman Estates, Ill.), and Carl Zeiss, (Zeiss Model LSM 5 Pascal; Carl Zeiss, Jena, Germany) are suitable for the methods of the present invention

In another embodiment, three dimensional arrays of the present invention are analyzed by two photon microscopy. Two photon microscopy utilizes a two photon effect, wherein a chromophore is excited by two low energy (infrared) photons, rather than by one higher energy (e.g. UV or visible light) photon. In two photon microscopy, the two photons are absorbed substantially contemporaneously (i.e. within 1 femtosecond or less). Because two photons are used to excite the sample, fluorescence depends on the square of the light intensity, and light intensity decreases approximately as the square of the distance from the focus. Because of the highly non-linear relationship between the light intensity and sample fluorescence, only those fluorescent species very near the focus of the beam are excited, and fluorophores above and below the plane of focus (i.e. other samples in the array) are not detected. Similar to confocal microscopy, a stack of two-dimensional images may be used to formulate a three dimensional image by sequentially changing the Z distance for measurement. In addition to two-photon excitation, excitation with more than two-photons is possible, for example three-photon excitation.

The analytical data from the array may also comprise other types of spectroscopic, imaging, sensor, or scanning data. Preferably, the data may also comprise measurements made using Raman, luminescence, ultraviolet-visible molecular absorbance, atomic absorbance, infra-red, near infrared, surface plasmon resonance, nuclear magnetic resonance, refractometry, interferometry, scattering, near-field scanning optical microscopy, laser-induced breakdown spectroscopy, ultrasonic spectroscopy, dielectric spectroscopy, microwave spectroscopy, resonance-enhanced multiphoton ionization, and the like. Combinations, improvements, and subclasses of these techniques can be used, for example surface plasmon resonance and fluorescence, Raman and infrared, and any others.

In an embodiment, more than one emission wavelength is collected per sample. This may be accomplished using techniques known in the art. For example, narrow bandpass interference filters may be used to select different emission wavelengths. This may be useful where emission profiles vary with sample composition, as when a fluorophore has a different emission profile depending on its molecular environment (e.g. whether the fluorophore is free in solution or incorporated in a polymer chain) or when samples in the array have more than one fluorophore.

In an embodiment, chemometric analysis may be employed. As defined herein, chemometrics is the science of relating measurements made on a chemical system or process to the state of the system via application of mathematical or statistical methods. For example, the data analysis system may monitor fluorescence correlated to at least one predetermined reaction component and use statistical and chemometrics techniques to correlate the fluorescence values to that sample component.

The methods used for data analysis may comprise either univariate or multivariate analysis. Thus, spectral data for each sample in the array may be monitored at one wavelength for univariate analysis, or at more than one wavelength for multivariate analysis. Generally, the spectra are analyzed using statistical techniques. Thus, the fluorescence values at a specific excitation wavelength may be analyzed using univariate linear regression calibration methods (see e.g. H. Mark and J. Workman, Statistics in Spectroscopy: Academic Press: San Diego, Calif., pp. 263-276 (1991); and J. C. Miller and J. N. Miller, Statistics for Analytical Chemistry, Ellis Horwood, New York, N.Y., pp. 101-139 (1993)). For example, univariate calibration models may be derived which provide quantitative prediction of the reaction product in samples of a three dimensional array based on fluorescence measurements at one wavelength.

In another embodiment, as where multiple parameters are measured, the methods used for data analysis may comprise multivariate analysis. For example, where the emission spectrum comprises several wavelengths or an entire fluorescence band, the characteristics of the sample may be determined using multivariate calibration algorithms such as Partial Least Squares Regression (PLS), Principal Components Regression (PCR), and the like (see e.g. Beebe, K. R., et al., 1998, Chemometrics: A Practical Guide, Wiley, New York, N.Y., pp. 183-339). Given a large enough span of calibration samples, multivariate calibration models are generally more robust than univariate models due to enhanced outlier detection capabilities and increased tolerance toward slight shifting in peak position or band shape. Also, multivariate calibration models allow for measurement of more than one variable or component of interest. PLS models may be used to correlate the sources of variation in the spectral data with sources of variation in the sample. Preferably, the PLS model is validated by statistical techniques. Such statistical techniques include, but are not limited to, leave one out cross-validation, venetian blinds, and random subsets (see e.g. Beebe, K. R., et al., 1998).

Thus, in an embodiment, a sample is irradiated with UV/visible light, and a reflection spectrum comprising a distinct range of wavelengths is monitored. In another embodiment, a sample is irradiated with UV/visible light and a fluorescence profile comprising a distinct range of wavelengths is monitored. By multivariate analysis, the presence and/or amount of multiple sample components is determined for each sample. Preferably, for both univariate and multivariate analysis, a sufficient number of known samples is used to generate the model such that the 95% confidence interval and the 95% prediction interval are suitable for routine screening of polymer production.

As an alternative to PLS modeling, chemometric analysis techniques such as principal component analysis (PCA) and related tools can be used to perform pattern recognition and/or to assess potential outliers in the analysis of one, or multiple, reaction components in each combinatorial event. PCA is used to identify a lower-dimensional coordinate system that captures the variance in a data set. The first principal component is the axis along the direction of the primary source of variation; the second principal component is the axis along the second most major source of variation; the third principal component is the axis along the third most major source of variation, and so forth.

Principal component scores for a spectrum may be computed by projecting the spectrum into a coordinate system which is defined by the major principal components calculated for a database of spectra. In an embodiment, plotting the spectral descriptors as a function of their principal component scores generates a two-dimensional spectral descriptor plot. The spectral descriptor plot provides for the direct comparison of all the spectra in the database. Because the position of each spectral descriptor in the plot is defined by the major components of variation in the spectra, spectra of similar shape will preferably generate spectral descriptors which fall near each other in the spectral plot. Conversely, spectra of dissimilar shape will preferably generate spectral descriptors which fall far from each other in the spectral plot.

As will be recognized by those of ordinary skill in the art, all or part of the steps in the method of the present invention may be coded or otherwise written in computer software, in a variety of computer languages including, but not limited to, C, C++, Pascal, Fortran, Visual Basic®, Microsoft Excel, MATLAB®, Mathematica®, Java, and the like. Accordingly, additional aspects of the present invention include computer software for performing one or more of the method steps set forth herein. The software code may be compiled and stored in executable form on computer readable media as, for example, computer ROM, floppy disk, optical disk, hard disks, cd ROM, or the like. The present invention may be further understood by reference to the following non-limiting example.

### EXAMPLE

Several arrays of polymer materials in the form of thin coatings were deposited onto a cylindrical dielectric waveguide with an outer diameter of approximately 2 mm. Each sample was approximately 0.75 mm diameter with approximately 0.5 mm spacing between elements. The polymer (polystyrene) included a fluorescent reagent (Nile red fluorophore) at a concentration of about 1 μM. FIG. 5 depicts the format of the array as a schematic diagram. The three dimensional libraries were evaluated using optical fluorescent microscopy. A confocal laser microscope (Zeiss Model LSM 5 Pascal; Carl Zeiss, Jena, Germany) was used to obtain fluorescence images of the 3D array. Images were collected using three laser wavelengths of (i.e. 488 nm, 514 nm and 543 nm) to increase the total excitation intensity and thereby reduce acquisition time. A pinhole size of 30-60 microns and a long-pass emission filter with a cut-off at 560 nm were employed, although pinhole sizes of ranging from 5 microns to 500 microns are suitable.

FIGS. 6 and 7 illustrate fluorescence confocal images of the 3D array probed at different spatial depths. Thus, in FIG. 6, samples at the lower surface of the cylinder are detected to the exclusion of samples on the upper and side surfaces. In FIG. 7, an image of the samples on the side of the waveguide are shown. In FIG. 8, samples from each surface of the waveguide are shown after three dimensional construction. In these experiments, a spacing of approximately 20 micrometers on the Z axis was used. For analysis, an average of two scans processed using the software supplied with the microscope was used.

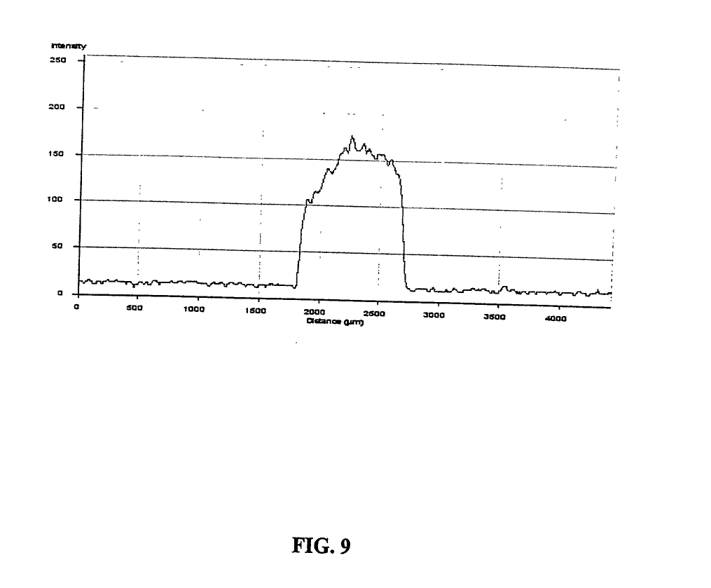

As shown in FIG. 8, which presents a combined fluorescence confocal image of a three dimensional array where elements on the distal, proximal, and side surfaces of a cylindrical array are probed, fluorescent confocal microscopy allow all of the samples in the array to be analyzed simultaneously. The data can be processed and stored for chemometric analysis using univariate or multivariate statistical techniques, e.g. PLS, PCR, or PCA as discussed herein, and others known in the art. Thus, FIG. 9 presents the fluorescence intensity map for the cross-section of one element probed along the line shown in FIG. 8. By compiling similar data for each sample of the array, the fluorescence map is transformed such that the data may be analyzed for single or multiple variables for each sample.

It will be recognized by those in the art that advantages of using three dimensional arrays for screening of combinatorial libraries and other types of sample analysis include:

1. The ability to include an increased number of samples per array;

2. The ability to provide a more dense arrangement of library elements per volume;

3. A higher throughput for performance testing of large numbers of samples;

4. A higher throughput for sample screening;

5. Flexibility in the generation of samples allowing for multiple combinations of reaction parameters over the 3D matrix;

6. Flexibility in the geometric presentation of samples for treatment and analysis; and

7. The ability to test a plurality of physical and chemical factors simultaneously in one screening.

It will be understood that each of the elements described above, or two or more together, may also find utility in applications differing from the types described herein. While the invention has been illustrated and described as a method for rapid, high-throughput, nondestructive analysis of combinatorial reactions, it is not intended to be limited to the details shown, since various modifications and substitutions can be made without departing in any way from the spirit of the present invention. For example, robotics equipment can be used to prepare the samples, and various types of parallel analytical screening methods can be incorporated. As such, further modifications and equivalents of the invention herein disclosed may occur to persons skilled in the art using no more than routine experimentation, and all such modifications and equivalents are believed to be within the spirit and scope of the invention as defined by the following claims.

What is claimed is:

1. A method for the generation and screening of three dimensional arrays comprising:
depositing a plurality of samples onto at least one substrate at discrete and defined positions in a three dimensional format such that each sample is isolated by the substrate from the other samples, and wherein each sample is defined by its (x, y, and z) coordinate to generate a three dimensional array of samples;
collecting analytical data from the sample array, wherein the analytical data is at least partially defined by its (x, y, and z) coordinate within the sample array;
correlating the analytical data collected from the array to the position of samples within the array; and
analyzing the analytical data for a parameter of interest.

2. The method of claim 1, wherein the array comprises multiple two dimensional arrays.

3. The method of claim 1, wherein the array comprises samples deposited on the surface of a three dimensional structure.

4. The method of claim 1, further comprising the use of masking and gradient deposition techniques to limit the deposit of samples to regions of interest.

5. The method of claim 3, further comprising evaporative methods for deposition of said samples.

6. The method of claim 5, wherein the evaporative methods comprise lasers, filaments, electron beams, or ion beams.

7. The method of claim 5, further comprising molecular beam epitaxy.

8. The method of claim 3, further comprising glow-discharge processes for deposition of said samples.

9. The method of claim 8, wherein the glow-discharge processes comprises sputtering.

10. The method of claim 3, further comprising chemical vapor deposition for deposition of said samples.

11. The method of claim 10, wherein the chemical vapor deposition process comprises photo-enhanced chemical vapor deposition or plasma-enhanced chemical vapor deposition.

12. The method of claim 3, further comprising pulsed-laser assisted deposition for deposition of said samples.

13. The method of claim 3, further comprising mechanical deposition of said samples.

14. The method of claim 13, wherein the mechanical deposition comprises spraying, spinning, dipping draining, flow coating, roller coating, pressure-curtain coating, or brushing.

15. The method of claim 3, wherein the samples have a thickness which ranges from 0.1 nm to 1 cm.

16. The method of claim 3, wherein the samples have a thickness which ranges from 1 nm to 1 mm.

17. The method of claim 3, wherein the samples have a thickness which ranges from 10 nm to 200 μm.

18. The method of claim 1, wherein the array comprises samples deposited within the interior of a three dimensional substrate.

19. The method of claim 18, wherein the samples are introduced into a pre-formed substrate.

20. The method of claim 18, wherein the samples are diffused into a pre-formed substrate.

21. The method of claim 18, wherein the substrate is tetrahedral.

22. The method of claim 18, wherein the substrate is polyhedral.

23. The method of claim 18, wherein the substrate is cylindrical.

24. The method of claim 18, wherein the substrate is spherical.

25. The method of claim 18, wherein the substrate is cubical.

26. The method of claim 18, wherein materials are diffused inwards from predetermined surface locations on the substrate.

27. The method of claim 26, wherein materials are diffused inwards from at least one surface plane of said substrate.

28. The method of claim 26, wherein materials are diffused inwards from at least one vertex of said substrate.

29. The method of claim 18, wherein at least some of the samples are introduced into a partially formed substrate and at least part of the substrate transformed to its final structure after deposition of the samples.

30. The method of claim 29, wherein the substrate comprises a gel which is polymerized upon application of radiation.

31. The method of claim 29, wherein the gel comprises acrylates or unsaturated polyester.

32. The method of claim 1, further comprising treating the deposited samples to initiate a chemical reaction within the three dimensional array.

33. The method of claim 32, wherein the treatment comprises the addition of a chemical agent.

34. The method of claim 33, wherein the treatment is applied differentially to at least some locations of the array.

35. The method of claim 33, wherein the treatment is applied in a constant manner to all locations of the array.

36. The method of claim 32, wherein the treatment comprises the application of electromagnetic radiation.

37. The method of claim 32, wherein the treatment comprises the application of ultrasound.

38. The method of claim 32, wherein the treatment comprises a change in temperature.

39. The method of claim 1, wherein the substrate is substantially inert.

40. The method of claim 1, wherein the substrate comprises a substance that interacts with at least some of the samples of the array.

41. The method of claim 1, wherein individual elements of the array are performance tested for stability upon exposure to at least one external agent.

42. The method of claim 41, wherein the external agent comprises a physical force.

43. The method of claim 41, wherein the external agent comprises electromagnetic radiation.

44. The method of claim 41, wherein the external agent comprises heat.

45. The method of claim 41, wherein the external agent comprises a chemical reagent.

46. The method of claim 41, wherein said external agent is applied in a constant manner to all samples of the array.

47. The method of claim 41, wherein the external agent is applied differentially to at least some of the individual samples in the array.

48. The method of claim 1, wherein spatially resolved measurement tools are used to collect and process data from said array.

49. The method of claim 48, further comprising applying a scanning probe over at least one of the samples.

50. The method of claim 49, wherein said scanning probe comprises confocal microscopy.

51. The method of claim 50, further comprising Raman confocal microscopy.

52. The method of claim 50, further comprising luminescence confocal microscopy.

53. The method of claim 49, wherein said scanning probe comprises two-photon microscopy.

54. The method of claim 49, wherein said scanning probe comprises multi-photon microscopy.

55. The method of claim 1, wherein collecting analytical data from the array is substantially simultaneous for each sample of the array.

56. The method of claim 1, wherein collecting analytical data from the array is performed separately for each sample of the array.

57. The method of claim 1, wherein the step of analyzing the collected data comprises univariate analysis.

58. The method of claim 1, wherein the step of analyzing the collected data comprises multivariate analysis.

59. A method for the generation and performance testing of three dimensional arrays comprising:
depositing a plurality of samples onto at least one substrate at discrete and defined positions in a three dimensional format such that each sample is isolated by said substrate from the other samples, and wherein each sample is defined by its (x, y, and z) coordinate to generate a three dimensional array of samples;
performance testing the array;
collecting data from the performance testing, wherein the data is at least partially defined by its (x, y, and z) coordinate within the sample array;
correlating the analytical data collected from the array to the position of samples within the array; and
analyzing the analytical data for a parameter of interest.

60. The method of claim 59, wherein the step of performance testing comprises applying at least one external agent to at least some of the samples of the array.

61. The method of claim 60, wherein the external agent comprises a physical force.

62. The method of claim 60, wherein the external agent comprises electromagnetic radiation.

63. The method of claim 60, wherein the external agent comprises heat.

64. The method of claim 60, wherein the external agent comprises a chemical reagent.

65. The method of claim 60, wherein said external agent is applied in a constant manner to all samples of the array.

66. The method of claim 60, wherein the external agent is applied differentially to at least some of the individual samples in the array.

67. A method for the analysis of three dimensional arrays comprising:
applying a scanning probe over a plurality of samples deposited onto at least one substrate at discrete and defined positions in a three dimensional format such that each sample is isolated by said substrate from the other samples, and wherein each sample is defined by its (x, y, and z) coordinate;
collecting analytical data from the array, wherein the analytical data is at least partially defined by its (x, y, and z) coordinate within the sample array;
correlating the analytical data collected from the array to the position of samples within the array; and
analyzing the analytical data for a parameter of interest.

68. The method of claim 67, comprising luminescent confocal microscopy.

69. The method of claim 67, comprising Raman confocal microscopy.

70. The method of claim 67, comprising two photon microscopy.

71. The method of claim 67, comprising multi-photon microscopy.
